(19) United States
(12) Patent Application Publication
(10) Pub. No.: US 2011/0276050 A1
(43) Pub. Date: Nov. 10, 2011

(54) STERNUM SUTURING STAPLE

(76) Inventors: Cesare Puricelli, Fagagna (IT); Gianni Ponte, Udine (IT)

(21) Appl. No.: 12/998,529

(22) PCT Filed: Oct. 29, 2009
(86) PCT No.: PCT/IB2009/054800

§ 371 Date: Jul. 18, 2011

(30) Foreign Application Priority Data
Oct. 30, 2008 (IT) MI2008A0001912

(51) Int. Cl. A61B 17/84

(52) U.S. Cl. 606/75

(57) ABSTRACT

A sternum suturing staple, comprising a central body (1) having at least two threaded portions (2, 2′) with opposite threads; two side members (6) each having a threaded part (7) adapted to couple to one of the central body threaded parts, and a claw (8) adapted to be located laterally to a sternum in an intercostal space, and in which the claws are adapted to cooperatively clamp the sternum so as to suture it, characterized in that the part or portion bearing the male thread has, adjacent and coaxial relative to said male thread, a non-threaded cylindrical stem length (5, 5′), while the part or portion bearing the female thread has, adjacent and coaxial relative to said female thread, a non-threaded cylindrical hole length (18) adapted to receive said stem length.

### FIELD OF INVENTION

The present invention concerns a staple for the suture of the sternum; it is of particular use after operations in which a sternotomy proved necessary, such as in operations of cardiac surgery.

### PRIOR ART

Currently, the sternotomy represents the most commonly practiced system of access to the thoracic cavity in operations like those of cardiac surgery. Closure of the sternum, at the end of the operation, is commonly performed by passing metal wires around the two halves into which it has been divided;

Thin bands with blocking device are also used.

However, this system presents most of the problems related to the use of metal wire, due to the stress which is necessary to pass it, the failed alignment of the sternum, the likelihood of severe infections, even lethal.

In order to replace these techniques, various types of staples have been proposed; those of the first type, made up by materials known as “memory shape alloys” are very expensive and they do not permit to adjust the closure tension.

WO 2004/016181 proposes a type of staple with two side members, adapted to be inserted into two holes, each produced on one of the two parts of the sternum and a screw device, adapted to draw them together or apart; the mechanism comprises a central body with two opposite threads which engage each with a corresponding thread on a claw. Rotating the central body the claws are drawn together or apart.

WO 2007/014910 proposes a similar type of staple, in which the claws are adapted to be inserted laterally to the sternum. In this document it is taught that the male threaded part is preferably located on the central body and that the female thread is produced on through holes adequately located on the elements which present the claws. This type of staple has solved most of the greater than detailed problems and it does not require, as in the previous case, a complex preparation of the sternum which potentially can weaken it.

However, considering the flexion stresses under which the staple goes during use, worsened by breathing, the mechanical resistance of the staple is not sufficient yet, and the mechanical failure risk is high. On the other hand it would not be desirable increasing the dimensions of the various components.

### SUMMARY

The problems discussed greater than have now been resolved according to the present invention by means of a new type of staple for the suture of the sternum, including:

A central body having at least two opposite threaded parts with opposite threads;

Two side elements each having a threaded part which couples to one of the threaded parts of the central body and one claw adapted to be positioned laterally to a sternum and in which the claws are adapted to cooperatively clamp the sternum so as to suture it, characterized in that the part or portion bearing the male thread has, adjacent and coaxial relative to said male thread, a non-threaded cylindrical stem length, while the part or portion bearing the female thread has, adjacent and coaxial relative to said female thread, a non-threaded cylindrical hole length adapted to receive said stem length.

Preferably, the male threads are on the central body, which has a cylindrical shape with a stem with the opposite male threads at the ends and, a non threaded stem length, the female threads being on the side member, in a preferably passing partially threaded hole with a non threaded length preferably facing the central body. In that case, the non-threaded length has a diameter equal or greater than the thread major diameter.

Preferably, said claw has one or more teeth in intermediate position adapted to be engaged with the sternum.

The claw preferably has a flat or concave side towards the position of the sternum, that is on the side of the central body.

### LIST OF THE FIGURES

The present invention will now be illustrated by the detailed description of a preferred, but not exclusive, embodiment, supplied purely by way of example, with the aid of the enclosed figures of which:

FIGS. 1 and 2 schematically represent a front view in partial section and a partial view of the lateral member of a staple according to the present invention

FIG. 3 schematically represents the central body of a staple according to the present invention

FIG. 4 schematically represents the central body of a staple according to the present invention in lateral view.

### DETAILED DESCRIPTION

With reference to FIG. 3, the central body 1 presents the threaded lengths 2 and 2′, one with a right thread, the other one with a left thread. According to a preferred embodiment of the invention, the body is substantially made up by a cylindrical stem and the threads of the portions 2 and 2′ are external threads (male). Preferably, a crown 3 is present, which can be knurled, or preferably present a gear tooth, as shown in FIG. 4, in order to ease the manual grip, or preferably with a dedicated device; the gear can also have a lobe-shape, to be engaged with a corresponding gear on the screw-driving apparatus.

Adjacent to the threaded lengths, which are at the ends of the stem, unthreaded cylindrical lengths 5 and 5′ are present, with diameter equal or greater than the external diameter of the threaded length.

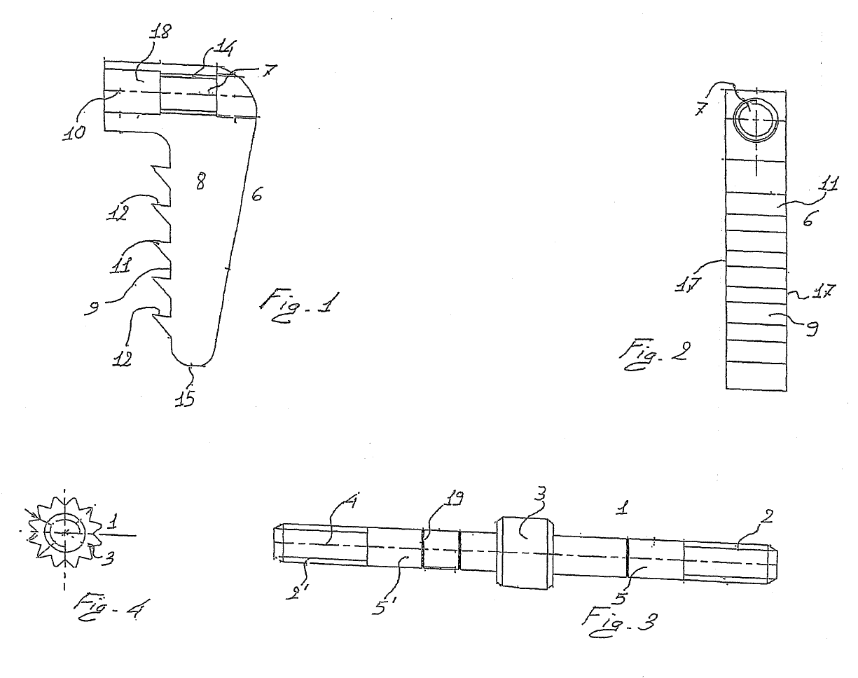

With reference to FIGS. 1 and 2, the threaded lengths are adapted to engage with the correspondent threaded lengths of the lateral elements of which one, with reference 6, is represented. Said elements present, according to a preferred embodiment of the invention, an internally threaded (female) hole 7, adapted to be engaged with the threaded lengths of the central body.

According to a preferred embodiment of the invention, the hole is through.

It is threaded only on a portion 14 of its length, for instance between 2 and 4 mm, the rest of the hole 18 being adapted both to host the threaded length of the central body without coupling with it, having a diameter equal or greater than that out of the threaded length, and the non-threaded cylindrical length 5, on an adequate length. That length can be chosen according to the requirements of mechanical resistance of the staple and of the overall resistance to screwing.

Preferably the diameter of the non threaded lengths, both on the central body, and on the side member are such as flexion stresses which are exerted to the staple during its function, can be exchanged between the relevant walls. Hence the nominal diameters will be equal or similar, preferably with a machining tolerance as to permit the mutual motion during the screwing or unscrewing of the threaded members, but such as not to prevent the exchanging of stresses. As a matter of fact it has been found, compared to the state-of-the-art staples, that the presence of cylindrical lengths, partially engaging as greater than described, provides an adequate mechanical resistance, compared to the case in which the stresses are exchanged only through the engaged threaded lengths.

Turning the central body around its longitudinal axis 4, it is caused a symmetrical motion of the side members that will get together or apart along the longitudinal axis 4, according to the rotation sense of the central body, thanks to the opposite threads.

With reference to FIG. 1 a side member is shown more in detail.

The side member 6, analogously to the opposite one (and symmetrical) presents a claw 8, adapted to be positioned laterally to the sternum in a inter-costal space. So the sternum can be positioned between the claws of the two side members of the staple. Getting together the side members, acting on the central body, the two halves of a sternum after median sternotomy can be drawn and clamped together, so performing the suture. Generally more than one staple will be necessary, in number variable according to the dimensions and the conditions of the sternum, for instance from four to six.

The shape of the claws can vary according to the needs and teeth can be present. For instance the shape shown in FIG. 1, has been deemed advantageous; it presents a side 9 substantially perpendicular to the axis 10 of the hole, side adapted to get in touch with the sternum; the side can also be inclined, for instance forming an acute angle with said axis and it can also be concave. It can present teeth 11, with adequate shape and dimensions, for instance, the side 12 of the teeth facing the side that presents the hole can be inclined as to improve the staple retention in position with regards to the sternum, for instance can be parallel to the axis of said hole, with the other side inclined as in figure.

Other shapes of the claw and the teeth are possible, for instance those described on WO 2007/014910. The free end 15 of the claw can be rounded in order to permit an insertion without trauma or lesions.

The other surfaces 17 bounding the side member (FIG. 2) can be, according to an embodiment of the invention, plane and parallel surfaces.

The dimensions can be unique or different dimensions can be considered for various types of sternum.

The tension of the staple can be given by means of a dedicated screwing device, for instance an apparatus like the one described in WO 2007/014910. The staple is dedicated to the sternum suture (human or also animal), in particular after sternotomy. The inter-costal spaces, in which the claws of the staple are to be inserted, can be surgically prepared according to known techniques, similar, for example to the ones used for the introduction of other types of suture systems.

The staple can be made up by any adequate material, preferably biocompatible, as the sternal suture is generally intended to be left permanently implanted in the patient. According to a preferred embodiment of the invention, the staple is totally made up of titanium.

The invention relates also a method for sternal suture comprising the phases of: staple insertion as greater than described, with the threaded lengths of the side members engaged with the corresponding threaded lengths of the central body, with the claws of the two side members in the inter-costal spaces astride the sternum, in particular when subjected to median sternotomy, and the rotation of the central body in as to match the two parts of the sternum and compress one onto the other with a pre-determined load, preferably by means of a motor-driven screwing device, more preferably with a dynamometric system. It is opportune that the non-threaded cylindrical stem length penetrates into the non threaded hole length on a length equal to at least 1.5 times the diameter size. There can be present marks, such as grooves 19 that permit the location of the screwing limits or in order to identify how to place the staple (right and left side) as to being screwed in a correct way without attempts, considering that in one sense the side members get together and in the opposite sense get apart, and it is not easy to recognize the correct sense, without such type of marks. A different colouring of the various parts can be presented, for instance a side member (right or left) can be coloured.

1. A sternum suturing staple, comprising:
a central body having at least two threaded portions with opposite threads;
two side members each having a threaded part adapted to couple to one of said central body threaded parts, and a claw adapted to be located laterally to a sternum, and in which said claws are adapted to cooperatively clamp the sternum so as to suture it, wherein the part or portion bearing said male thread has, adjacent and coaxial relative to said male thread, a non-threaded cylindrical stem length, while the part or portion bearing said female thread has, adjacent and coaxial relative to said female thread, a non-threaded cylindrical hole length adapted to receive said stem length.

2. The staple according to claim 1, wherein said claw has one or more teeth adapted to engage with the sternum.

3. The staple according to claim 2, wherein said claw has a rounded free end.

4. The staple according to claim 3, wherein said central body has a crown adapted to be rotated about a longitudinal axis of said central body, manually or by means of a special apparatus.

5. The staple according to claim 4, wherein said crown is toothed and gear-shaped.

6. The staple according to claim 5, made of biocompatible material.

7. The staple according to claim 6, made of titanium.

8. The staple according to claim 6, wherein said male threads are on said central body, which has a cylindrical shape with a stem with the opposite threads at the ends and, a non-threaded stem length, the female threads being on the side member, in a partially threaded hole with a non-threaded length (18) preferably facing the central body.

9. The staple according to claim 8 wherein said non-threaded length has a diameter equal to or greater than said thread major diameter.

10. The staple according to claim 1, wherein the diameter of said non-threaded cylindrical length and of said non-threaded cylindrical hole are essentially the same, with such a clearance as to allow threading the threaded parts without resistance.

11. A sternum suturing staple, comprising:
a central body having at least two threaded portions with opposite threads;
two side members each having a threaded part adapted to couple to one of said central body threaded parts, and a claw having one or more teeth and a rounded free end, said claw being adapted to be located laterally to a sternum, and in which said claws are adapted to cooperatively clamp the sternum so as to suture it, wherein the part or portion bearing said male thread has, adjacent and coaxial relative to said male thread, a non-threaded cylindrical stem length, while the part or portion bearing said female thread has, adjacent and coaxial relative to said female thread, a non-threaded cylindrical hole length adapted to receive said stem length.

12. A titanium sternum suturing staple, which comprises:
a central body having at least two threaded portions with opposite threads, said central body having a crown adapted to be rotated about a longitudinal axis of said central body,
two side members each having a threaded part adapted to couple to one of said central body threaded parts, and a claw adapted to be located laterally to a sternum, and in which the claws are adapted to cooperatively clamp the sternum so as to suture it, wherein the part or portion bearing the male thread has, adjacent and coaxial relative to said male thread, a non-threaded cylindrical stem length, while the part or portion bearing said female thread has, adjacent and coaxial relative to said female thread, a non-threaded cylindrical hole length adapted to receive said stem length.
